(19) United States
(12) Patent Application Publication
(10) Pub. No.: US 2018/0168409 A1
(43) Pub. Date: Jun. 21, 2018

(54) TEXTURED MICROFIBER FOR SKIN-TIGHTENING FILM REMOVER

(71) Applicant: L'Oreal, Paris (FR)

(72) Inventors: Alexandra Jane Elisa Farran, Dayton, NJ (US); Tsung-Hui Marisal Mou, Towaco, NJ (US)

(73) Assignee: L'Oreal, Paris (FR)

(21) Appl. No.: 15/383,864

(22) Filed: Dec. 19, 2016

(51) Int. Cl. A47K 7/03; A61Q 1/14; A61K 8/02; B08B 1/00; B08B 3/04

(52) U.S. Cl. CPC A47K 7/03; A61Q 1/14; A61K 2800/87; B08B 1/001; B08B 3/04; A61K 8/0208

(57) ABSTRACT

Articles having microfiber exteriors are wetted with a removal formulation to remove long-wearing cosmetic films from facial features. Films include skin tightening films. Articles include mittens. Removal formulations include water-based, oil-based, or biphasic removal formulations.

### SUMMARY

An aspect of this disclosure is related to a method for the removal of a film adhered to skin, in which the method includes rubbing the film with a microfiber article wetted with a removal formulation.

In an embodiment, the method further comprises soaking the film for period of time with the wetted microfiber article before rubbing.

In an embodiment, soaking comprises about 30 seconds or less.

In an embodiment, the microfiber article is a mitten.

In an embodiment, the removal formulation is an oil-based formulation

In an embodiment, the removal formulation is a water-based formulation.

In an embodiment, the removal formulation is a biphasic formulation comprising an oil and water phase.

In an embodiment, the film is a skin tightening formulation.

In an embodiment, the film is adhered on a facial feature.

In an embodiment, the facial feature is on or near the eye.

In an embodiment, rubbing comprises rubbing for under 2 minutes to remove the film.

An aspect of this disclosure is related to a combination of an article and formulation, in which the article has at least one exterior surface section made of microfibers and the removal formulation is configured for removing a film adhered on skin.

In an embodiment, the article is a mitten.

In an embodiment, the exterior of the article comprises a fabric made from microfibers of less than 1 denier.

In an embodiment, the interior of the article is lined with an impermeable layer.

In an embodiment, the exterior of the article comprises a terry cloth fabric made from microfibers of less than 1 denier.

In an embodiment, the exterior of the article is made from microfibers that have a cross sectional shape having corners.

In an embodiment, the exterior of the article is made from polyester or polyamide or both polyester and polyamide microfibers.

In an embodiment, the removal formulation is an oil-based formulation.

In an embodiment, the removal formulation is a water-based formulation.

In an embodiment, the removal formulation is a biphasic formulation having an oil and water phase.

In an embodiment, the combination further comprises a package containing the microfiber article and the removal formulation.

This summary is provided to introduce a selection of concepts in a simplified form that are further described below in the Detailed Description. This summary is not intended to identify key features of the claimed subject matter, nor is it intended to be used as an aid in determining the scope of the claimed subject matter.

### DESCRIPTION OF THE DRAWINGS

The foregoing aspects and many of the attendant advantages of this invention will become more readily appreciated as the same become better understood by reference to the following detailed description, when taken in conjunction with the accompanying drawings, wherein:

FIG. 1 is a diagrammatical illustration of a splittable microfiber;

FIG. 2 is a diagrammatical illustration of a splittable microfiber;

FIG. 3 is a diagrammatical illustration of a mitten made from microfibers;

FIG. 4 is a diagrammatical illustration of a mitten made from microfibers;

FIG. 5 is a diagrammatical illustration of a mitten made from microfibers;

FIG. 6 is a diagrammatical illustration of a mitten made from microfibers;

FIG. 7 is a diagrammatical illustration of a wedge made from microfibers; and

FIG. 8 is a diagrammatical illustration of a wipe made from microfibers.

### DETAILED DESCRIPTION

Lotions, cosmetics, and other skin treatments are worn for an extended period of times as films. Long wearing films are used cosmetically on facial features for a variety of purposes, such as, to tighten the skin, to smooth out wrinkles, to moisturize the skin, and as cosmetics. A problem often encountered is the removal of the long wearing film. Films can adhere stubbornly to skin. In order to remove such films requires aggressive rubbing or a prolonged period or both prolonged and aggressive rubbing. The aggressive or prolonged rubbing can lead to irritation of the skin. Furthermore, even after prolonged or aggressive rubbing, film residues may remain on the skin. Accordingly, in an aspect of this disclosure, an article is disclosed that when used in combination with a removal formulation will assist in efficiently and gently removing films from facial features and other areas of the skin.

Film forming polymers for application on the skin are disclosed in U.S. Pat. No. 8,771,656, incorporated herein expressly by reference. In an embodiment, a film includes anionic copolymers based on methacrylic acid and methyl methacrylate in a random, block, or alternating configuration. In U.S. Pat. No. 9,050,474, incorporated herein expressly by reference, a class of polymers known as organosiloxanes is disclosed as film forming polymers for application on the skin. In an embodiment, organosiloxanes include polydimethylsiloxane (PDMS or Dimethicone). Additionally, in an embodiment, silicone polymers are copolymerized with other polymers, such as polyurethanes and ethylenically unsaturated monomers or polymers thereof. U.S. Pat. No. 8,277,791, incorporated herein expressly by reference, discloses film forming polymers include silicone polyurethane polymers in combination with at least one elastomer selected from silicone gums, polyisobutylene, natural rubbers, and block-copolymer rubbers.

In an embodiment, the film composition is a skin tightening composition. In an embodiment, skin tightening film compositions are disclosed in WO 2016/100742 and WO 2016/100690, both incorporated herein expressly by reference. In an embodiment, a skin tightening film composition includes an elastomer, an adhesive film forming polymer, and a filler. In an embodiment, a skin tightening composition includes at least one thermoplastic elastomer chosen from amorphous hydrocarbon block copolymers of styrene and monomers of hydrocarbon containing 2 to 5 carbon atoms and comprising one or two ethylenic unsaturations, and having a first T_{g} below about 0° C., and a second T_{g} greater than about 25° C.; at least one adhesive film-forming polymer chosen from polymer particles of C₁-C₄ alkyl(methacrylate)polymer, stabilized in a nonaqueous dispersion; and at least one filler. In an embodiment, the thermoplastic elastomer includes one or more styrene blocks and one or more blocks of units selected from butadiene, ethylene, propylene, butylene, isoprene, or mixtures thereof. In an embodiment, the thermoplastic elastomer is a diblock copolymer chosen from styrene-ethylene/propylene copolymers, styrene-ethylene/butadiene copolymers, styrene-ethylene/butylene copolymers, styrene-butadiene, or styrene-isoprene copolymers; a triblock copolymer chosen from styrene-ethylene/propylene-styrene copolymers, styrene-ethylene/butadiene-styrene copolymers, copolymers of styrene-isoprene-styrene, or copolymers of styrene-butadiene-styrene; or mixtures thereof. In an embodiment, adhesive polymers include C₁-C₄ alkyl (meth)acrylate and ethylenically unsaturated acid monomer of C₁-C₄ alkyl(methacrylate) polymer in an oil dispersion. In an embodiment, the C₁-C₄ alkyl(methacrylate)polymer is chosen from methyl(meth)acrylate, ethyl (meth)acrylate, n-propyl (meth)acrylate, isopropyl (meth)acrylate, n-butyl (meth)acrylate and tert-butyl (meth)acrylate polymers. In an embodiment, the oil is a hydrocarbon based oil of up to 40 carbon atoms, such as isododecane. In an embodiment, the filler is chosen from silica particles, hydrophobic silica aerogel particles, or aerogel particles of hydrophobic silica surface-modified with trimethylsilyl groups. In an embodiment, film-forming compositions include volatile organic solvents, volatile hydrocarbon-based oils, or volatile silicone oils. In an embodiment, the solvent is chosen from branched C₈ to C₁₆ alkanes, C₈ to C₁₆ isoalkanes, isododecane, isodecane, isohexadecane, octamethyltetrasiloxane, decamethylcyclo-pentasiloxane, dodecamethylcyclohexasiloxane, heptamethyloctyltrisiloxane, hexamethyldisiloxane, decamethyltetrasiloxane, dodecamethylpentasiloxane, and mixtures thereof. In an embodiment, silicone elastomers are chosen from silicone crosspolymers, such as dimethicone crosspolymers, dispersed in at least one oil.

In an embodiment, a film composition and removal formulation are disclosed in U.S. application Ser. Nos. 15/087,115 and 15/094,259, entitled, “Systems and Methods for Improving the Appearance of the Skin” and “Compositions for Removing Cosmetic Films,” both are incorporated herein expressly by reference. In an embodiment, a film composition includes one or more thermoplastic elastomers chosen from amorphous hydrocarbon block copolymers of styrene and monomers of hydrocarbon containing 2 to 5 carbon atoms and having one or two ethylenic unsaturations, and having a first T_{g} (glass transition temperature) below about 0° C., and a second T_{g} greater than about 25° C.; one or more adhesive film-forming polymers chosen from polymer particles of C₁-C₄ alkyl(methacrylate)polymer, stabilized in a non-aqueous dispersion; and at least one filler; and a cosmetic composition for making up the skin, comprising at least one organic pigment and optionally at least one volatile solvent. In an embodiment, the cosmetic composition for making up the skin includes organic pigments chosen from nitroso, nitro, azo, xanthene, pyrene, quinoline, anthraquinone, triphenylmethane, fluorane, phthalocyanin, metal complex, isoindolinone, isoindoline, quinacridone, perinone, perylene, diketopyrrolopyrrole, indigo, thioindigo, dioxazine, triphenylmethane and quinophthalone compounds.

The film forming compositions explicitly recited are not meant to be exhaustive, but merely are provided to give exemplary films that are pertinent to this disclosure. In an embodiment, the methods and articles of this disclosure are applicable to removal of all types of films adhered to skin, facial features, and body features.

In addition to the film forming polymers, the film compositions include any number of additional ingredients depending on the purpose of the film. In an embodiment, the film includes one or more cosmetic ingredients selected from humectants emollients, moisturizers, skin-tightening ingredients, anti-wrinkle ingredients, concealers, matte finishing agents, pigments, colorants, proteins, anti-oxidants, bronzers, chelating agents, emulsifiers, ultraviolet (UV) absorbing agents, oil absorbing agents, anti-foam agents, anti-tack agents, thickeners, fragrances, preservatives, anti-microbials, fungistats, neutralizing agents, vitamins, plasticizers, cohesion agents, basifying and acidifying agents, fillers, solvents, and combinations thereof.

In an embodiment, a film is a film that is adhered to facial features, including around the eyes, lips, cheeks, forehead, neck area. In an embodiment, a film is a film that is adhered on the skin.

In an embodiment, a film is a skin tightening film. In an embodiment a film is a long wearing film, also known as a long lasting film. In an embodiment, a long wearing or long lasting film is any film that is worn for up to or more than 10 minutes. In an embodiment, a long wearing film is any film that is worn for up to or more than 20 minutes. In an embodiment, a long wearing film is any film that is worn for up to or more than 30 minutes. In an embodiment, a long wearing film is any film that is worn for up to or more than 1 hour. In an embodiment, a long wearing film is any film that is worn for up to or more than 2 hours. In an embodiment, a long wearing film is any film that is worn for up to or more than 4 hours. In an embodiment, a long wearing film is any film that is worn for up to or more than 6 hours. In an embodiment, a long wearing film is any film that is worn for up to or more than 12 hours. In an embodiment, a long wearing film is any film that is worn for up to or more than 24 hours.

In an aspect of this disclosure, a removal formulation is used in conjunction with a microfiber article for the removal of films adhered on skin or other integuments. In an embodiment, a microfiber article is wetted with a removal formulation, and then, the microfiber article is rubbed or otherwise applied against the film to aid in removal of the film. In an embodiment, before rubbing, the wetted microfiber article is dabbed or applied on the skin in a manner to soak the film. In an embodiment, the wetted microfiber article is dabbed or applied on the skin in a manner to soak the film for less than or up to 30 seconds. In an embodiment, the wetted microfiber article is dabbed or applied on the skin in a manner to soak the film for up to 60 seconds. In an embodiment, rubbing or application is done manually with an article, such as with a wearable mitten. In an embodiment, rubbing or application is done mechanically, such as with a rotating or vibrating handheld appliance.

Removal formulations include removal formulations that are based on oil and removal formulations that are based on water, or both water and oil. In an embodiment, a removal formulation is based on both an oil and water phase. In an embodiment, oil based removal formulations include vaseline oil which may be mixed with fatty esters. In an embodiment, water based removal formulation contain an aqueous solution of a surfactant.

In an embodiment, removal formulations include biphasic removers. Conventional biphasic removers include, for example, Bi-Facil™ by Lancome.

Based on the type of film being removed, being either an oil based, a water based, or a biphasic based removal formulation, the removal formulation is selected that is appropriate to the film. In an embodiment, an oil based removal formulation is used to remove oil based films. In an embodiment, a water based removal formulation is used to remove water based films. In an embodiment, a biphasic removal formulation is used to remove either water based or oil based films.

Removal formulations are described, for example, in U.S. Pat. No. 5,165,917, incorporated herein expressly by reference.

In an embodiment, a biphasic removal formulation has an aqueous phase containing at least one surfactant and an oily phase having at least one cosmetic oil. In an embodiment, a biphasic removal formulation has an oil phase to water phase weight ratio ranging from anyone of 10:90 to 90:10.

In an embodiment, a removal formulation has a water phase including demineralized water, or a scented water, such as rosewater.

In an embodiment, a removal formulation includes anionic surfactants, nonionic surfactants, or amphoteric surfactants.

In an embodiment, a removal formulation includes a nonionic surfactant selected from, but not limited to, fatty esters of polyoxyethylene sorbitol, fatty polyoxyethylene alcohols, polyoxyethylene alkylphenols, and ethylene oxide and propylene oxide condensates.

In an embodiment, a removal formulation includes anionic surfactants selected from, but not limited to, alkyl ether sulfates, alkyl sulfoacetates, alkyl sulfosuccinates, alkylamidosulfosuccinates, alkylamidopolypeptides, and acylsarcosinates.

In an embodiment, a removal formulation includes amphoteric surfactants selected from, but not limited to, alkylamidopropyl dimethylbetaines, alkylamidobetaines, imidazoline derivatives, N-alkyl beta-imino-dipropionates.

In an embodiment, the oily phase of a removal formulation includes a mixture of oils, such as mineral oil, vegetable oils, synthetic oils, or silicone oils. In an embodiment, mineral oils include, but are not limited to, vaseline oil and higher aliphatic hydrocarbons, such as isohexadecane. In an embodiment, vegetable oils include, but are not limited to jojoba oil and safflower oil. In an embodiment, synthetic oils include, but are not limited to, alkyl palmitates having 2 to 10 carbon atoms, such as isopropyl palmitate or 2-ethylhexyl palmitate, and alkyl adipates with 2 to 10 carbon atoms, such as di(2-ethylhexyl) adipate. In an embodiment, silicone oils include, but are not limited to, cyclopentadimethylsiloxane.

In an embodiment, the removal formulation includes a silicone oil-based biphase formulation without surfactants.

In an embodiment, a film composition and removal formulation are disclosed in U.S. application Ser. Nos. 15/087,115 and 15/094,259, entitled, “Systems and Methods for Improving the Appearance of the Skin” and “Compositions for Removing Cosmetic Films,” both of which are incorporated herein expressly by reference. In an embodiment, the removal formulation includes a solvent chosen from at least one oil with solvent properties. In an embodiment, the at least one solvent includes one or more low-viscosity, low-volatility, linear and/or cyclic silicone oils. In an embodiment, the at least one solvent includes one or more low-viscosity, low-volatility, linear and/cyclic silicone oils. In an embodiment, the at least one solvent consists essentially of one or more low-viscosity, low-volatility, linear and/or cyclic silicone oils. In an embodiment, the removal formulation includes a solvent having at least one low-viscosity, low-volatility silicone oil chosen from (a) cyclic silicone oils, and (b) linear silicone oils of formula: R₃SiO—(R₂SiO)ₙ—SiR₃ wherein: R, which may be identical or different, is chosen from: a saturated or unsaturated hydrocarbon-based radical, containing from 1 to 10 carbon atoms, optionally substituted with one or more fluorine atoms or with one or more hydroxyl groups, or a hydroxyl group, one of the radicals R optionally being a phenyl group, and n is an integer ranging from 0 to 8; wherein the at least one low-viscosity, low-volatility silicone oil is present in the composition in an amount of at least about 20% by weight, relative to the composition, and wherein the at least one low-viscosity, low-volatility silicone oil has a viscosity of less than about 350 cSt. In an embodiment, the removal formulation includes water of at least about 50% by weight, relative to the composition. In an embodiment, the removal formulation includes at least one emulsifier and wherein the composition is in the form of a water-in-oil emulsion. In an embodiment, the at least one low-viscosity, low-volatility silicone is present in an amount of at least about 40% by weight, relative to the composition. In an embodiment, the removal formulation has water in an amount of at least about 40% by weight, relative to the composition, wherein the composition is in the form of a biphase composition. In an embodiment, the removal formulation includes at least one wax in an amount of at least about 40% by weight, relative to the composition, wherein the composition is in the form of a solvent-wax dispersion. In an embodiment, the removal formulation includes additional components chosen from preservatives, clarifiers, emulsifiers, surfactants, pH adjusting agents, antioxidants, fragrances, colorants such as soluble dyes and pigments, optical brighteners, electrolytes, stabilizers, plant extracts, proteins, amino acids, vitamins, glycols, emollients, and mixtures thereof.

In an embodiment, the removal formulation additionally contains conventional cosmetic adjuvants which may be in either phase according to whether they are hydrophilic or lipophilic. In an embodiment, the removal formulation includes one or more of perfumes, preservatives, dyes, softening agents, buffers, moisteners, and electrolytes.

The removal formulations explicitly recited are not meant to be exhaustive, but merely are provided to give exemplary removal formulations that are pertinent to this disclosure. In an embodiment, the methods and articles of this disclosure are applicable with all types of removal formulations, regardless whether the formulations are based on water, oil, or are biphasic.

In an aspect of this disclosure, articles have an exterior made from microfibers. In an embodiment, a microfiber is an ultrafine filament of less than about 1 denier. In an embodiment, a microfiber is an ultrafine filament of less than about 3 denier. In an embodiment, a microfiber is an ultrafine filament of less than about 5 denier. In an embodiment, a microfiber is an ultrafine filament of less than about 10 denier. A denier is a unit defining the fineness of single fibers in terms of liner mass density, i.e., the weight of a given length of fiber. A denier equals one gram for every 9000 meters.

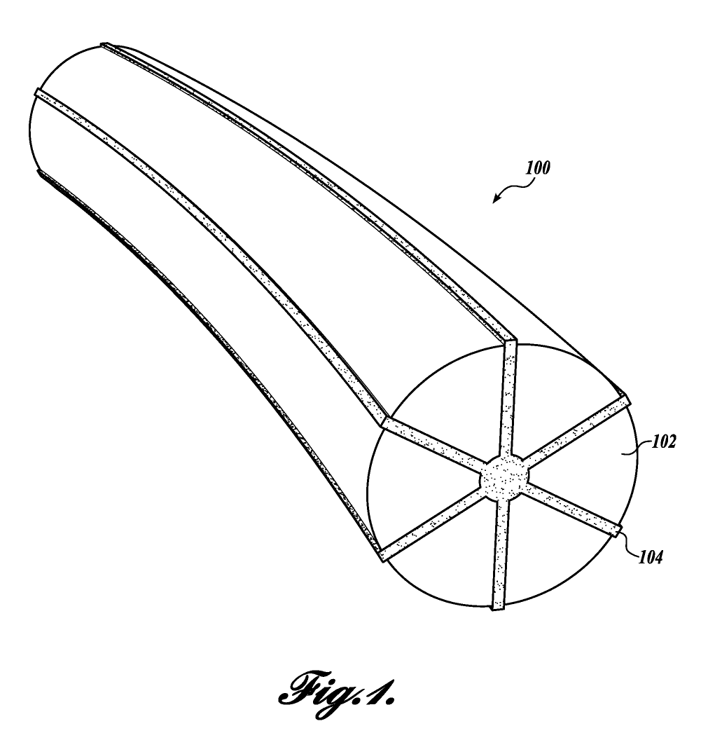

In an embodiment, microfibers have noncircular cross sectional shapes. In an embodiment, microfibers have a wedge or triangular cross sectional shapes. In an embodiment, microfibers with geometrical shapes are made, in an embodiment, from “splittable” microfibers. An embodiment of a splittable microfiber is represented by the diagrammatical illustration of FIG. 1. FIG. 1 shows the cross section of a splittable microfiber 100 that has wedges 102 interspersed between arms 104 extending radially from a center. The shape is sometimes described as an asterisk or star. In an embodiment, the microfiber shape represented by FIG. 1 is made from a polyamide and a polyester, wherein the polyester forms the wedges 102 and the polyamide forms the asterisk 104. In an embodiment, the wedges 104 are splittable from the asterisk shape. FIG. 2 shows another embodiment of a splittable microfiber 200 shown to contain wedges 202, 204. In an embodiment, the microfibers are segmented, meaning that each of the wedges is a different material than the adjoining wedges. For example, in an embodiment, the segments of FIG. 2 alternate between polyester and polyamide. However, splittable microfibers are also made from a single polymer. While FIGS. 1 and 2 show splittable microfibers with a generally circular cross section, other splittable microfibers have been made with other cross sections that are noncircular.

In an embodiment, the splittable microfibers are about 3, 5, or 10 deniers or less. In an embodiment, the splittable microfibers are carded to form a web, and then the web passes under hydroentangling jets, which split the fibers into individual wedges and entangle the fibers to give the fabric its feel and strength. As a result, the fabric contains microfibers down to 1.0 denier or less per filament.

In an embodiment, the splittable microfibers are made from a first polymer and a second soluble polymer. For example, in an embodiment, splittable microfibers are made from a polyester and co-polyester, where the polyester exists as microfiber islands in a sea of co-polyester. Then, the fibers are chemically treated to dissolve the co-polyester leaving the polyester microfibers.

In an embodiment, the splittable microfibers are made by meltblowing or spinning two or more dissimilar materials, such as polyester and polyamide, through a specialized die. Once the splittable microfiber is spun, the microfibers are processed, such as by carding, to be used as for making woven fabrics, knit fabrics, and the like. In addition, microfibers are also made into non-woven fabrics. In an embodiment, before or after carding, the microfibers are subjected to mechanical action or chemicals to cause the individual segments of the splittable microfiber to separate. The splitting of the splittable microfibers into even smaller microfibers results in a very soft fabric. In an embodiment, the split microfibers entangle with each other resulting in the very soft feel. Further, the cross sectional geometry of the split microfibers give microfibers certain advantages. In an embodiment, for example, the microfibers 100, 200 have cross sectional shapes that include sharp edges or corners. In an embodiment, these microfiber shapes enhance the removal properties of fabrics made from such microfibers. In an embodiment, an article has an exterior made from splittable microfibers. In an embodiment, an article has an exterior made from the split microfibers that have been split from a splittable microfiber.

The list of polymers explicitly recited for making microfibers are not meant to be exhaustive, but, merely are provided to give exemplary microfibers that are pertinent to this disclosure. In an embodiment, the methods and articles of this disclosure are applicable to all types of microfibers, whether synthetic or natural.

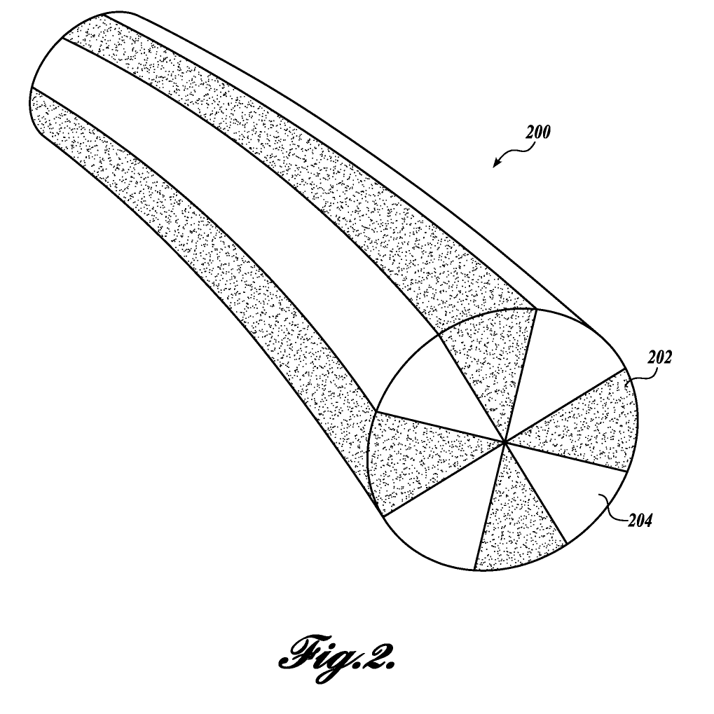

In an embodiment, film removal articles are made from microfiber fibers. FIGS. 3-6 show mittens that have at least an exterior made from microfibers. The mittens are used in combination with a removal formulation to remove films. Specifically, the microfiber exterior is wetted with a removal formulation. Then, the article is used to soak the film for a period of time, followed by rubbing. FIG. 3 is a diagrammatical illustration of a mitten 300 that has at least one exterior surface section made from microfibers. In an embodiment, an entire side or both sides are made from microfibers. The mitten 300 has a separate pocket for the thumb, and a separate pocket for the remaining four fingers. FIG. 4 is a diagrammatical illustration of a mitten 400 that has at least one exterior surface section made from microfibers. In an embodiment, an entire side or both sides are made from microfibers.

In an embodiment, the film removal articles include one or more hydrophobic materials, hydrophilic materials, lipophilic materials, and the like. For example, in an embodiment, the film removal articles include one or more hydrophilic materials capable of drawing moisture, polar formulations, skin tightening film formulations, film-forming formulations, film forming polymers, and the like from the surface of the skin. In an embodiment, the film removal articles include one or more hydrophobic materials capable of drawing film-forming formulations, skin tightening film formulations, film forming polymers, and the like from the surface of the skin.

In an embodiment, film removal articles include one or more woven microfiber fabric layers.

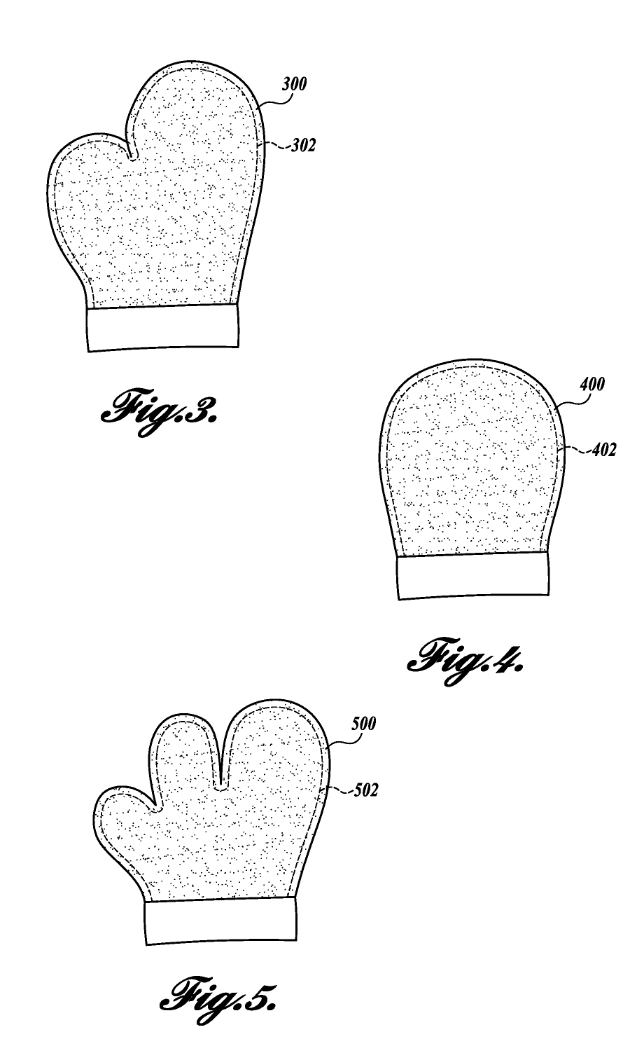

The mitten 400 has a separate pocket for any number of fingers including the thumb. In an embodiment, the mitten 400 of FIG. 4 is used over four fingers or less, for example. In an embodiment, the mitten 400 of FIG. 4 is used over three fingers. In an embodiment, the mitten 400 of FIG. 4 is used over two fingers. FIG. 5 is a diagrammatical illustration of a mitten 500 that has at least one exterior surface section made from microfibers. In an embodiment, an entire side or both sides are made from microfibers. The mitten 500 has a separate pocket for the thumb, a separate pocket for the index finger, and a separate pocket for the remaining three fingers. The embodiment of FIG. 5 allows the thumb and index finger to cooperate for easily grasping objects. FIG. 6 is a diagrammatical illustration of a mitten 600 that has at least one exterior surface section made from microfibers. In an embodiment, an entire side or both sides are made from microfibers. The mitten 600 has a separate pocket for the thumb, a separate pocket for the index finger, a separate pocket for the middle or ring finger, and a separate pocket for the little or pinkie finger. In the embodiment of FIG. 6, each of the fingers moves separately, which may be advantageous to allow control over the rubbing or buffing action. In an embodiment, the mittens shown in FIGS. 3-6 include an elastic band to secure the mitten to the wrist, or to any other part of the arm or the hand. In an embodiment, the mittens shown in FIGS. 3-6 include an exterior woven fabric comprised of microfibers. In an embodiment, the mittens shown in FIGS. 3-6 include a terry cloth exterior fabric comprised of microfibers. In an embodiment, the mittens shown in FIGS. 3-6 include an interior impermeable layer shown as 302, 402, 502, and 602, such layer being impermeable to at least water.

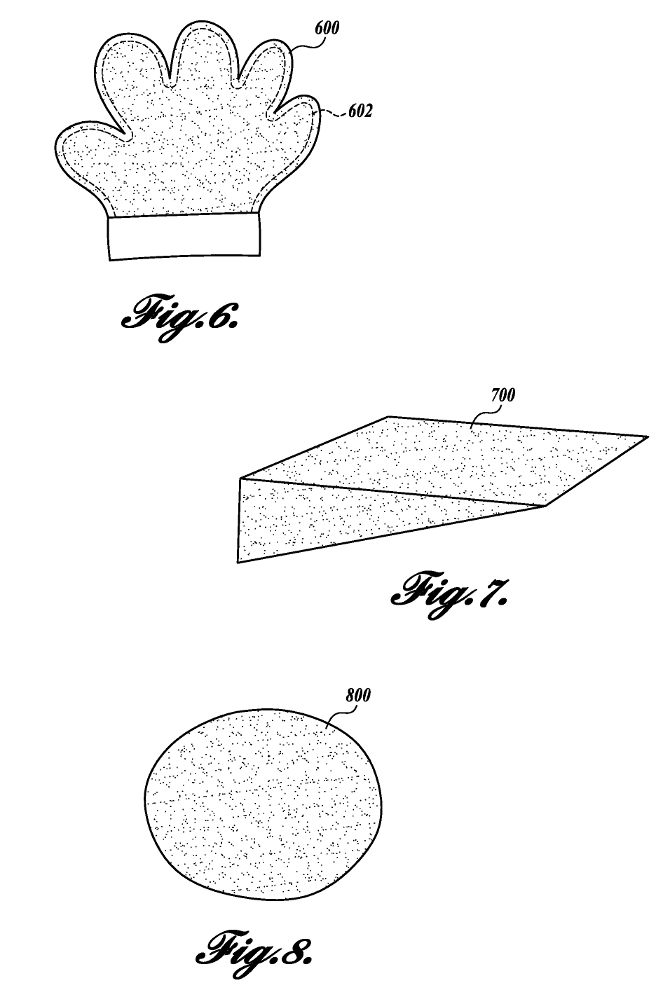

Film removal articles are not limited to mittens. Referring to FIG. 7, a microfiber fabric covered wedge 700 is illustrated. Referring to FIG. 8 a microfiber fabric wipe 800 is illustrated. The wedge 700 and wipe 800 have at least one exterior side section made from microfibers.

In an aspect of this disclosure, a microfiber article wetted or soaked in a removal formulation is used to remove a film. In an embodiment, the film is a skin tightening film.

In an embodiment, the unique microfiber cross sectional geometry in combination with a removal formulation allows for an efficient yet gentile removal of skin tightening films, in comparison to cotton pads, sponges, wipes, cotton balls, and the like. Non-microfiber articles can lead to skin irritation due to prolonged or harsh rubbing on the skin. In an embodiment, the combination of a microfiber article and a film removal formulation are sold together as a unit.

### Example

Experimental Detail:

Consumers used a microfiber mitten in accordance with embodiments of the invention and a round cotton pad with a biphasic make-up remover to remove the skin tightening film after 6 hours of wear. Enough of the remover solution was deposited on the microfiber mitten or the cotton pad to saturate the fabric. Then the saturated fabric was applied to the skin tightening film and dabbed on for about 30 sec before starting to rub off the skin tightening film. The removal time was recorded, including the 30 seconds of soaking time.

Results:

| | | Mitten + biphasic | | Cotton pad + biphasic | |
|---|---|---|---|---|---|
| | | make-up remover | | make-up remover | |
| | | STUDY 1 | | STUDY 2 | |
| Panelist #1 | R eye | 2 | min | 2 | min |
| | L eye | 1.25 | min | 2 | min |
| Panelist #2 | R eye | 1.30 | min | NA | |
| | L eye | 1.15 | min | NA | |
| Panelist #3 | R eye | 1.10 | min | 3 | min |
| | L eye | 1.20 | min | 2.30 | min |
| Panelist #4 | R eye | 40 | sec | 1.15 | min |
| | L eye | 20 | sec | 1.45 | min |
| Panelist #5 | R eye | 1.10 | min | 1.30 | min |
| | L eye | 55 | sec | 1.30 | min |
| AVG | R eye | 78 | sec | 116 | sec |
| | L eye | 63 | sec | 116 | sec |

| | | Mitten + biphasic | |
|---|---|---|---|
| STUDY 3 | | make-up remover | |
| Panelist #1 | R eye | 45 | sec |
| | L eye | 45 | sec |
| Panelist #2 | R eye | 2.30 | min |
| | L eye | 1.50 | min |
| Panelist #3 | R eye | 1.15 | min |
| | L eye | 50 | sec |
| Panelist #4 | R eye | 1 | min |
| | L eye | 1.30 | min |
| Panelist #5 | R eye | 1.30 | min |
| | L eye | 1.30 | min |
| AVG | R eye | 84 | sec |
| | L eye | 61 | sec |

The results show that a microfiber mitten wetted with the biphasic remover was more efficient in terms of time to remove the films as compared with non microfiber cotton.

While illustrative embodiments have been illustrated and described, it will be appreciated that various changes can be made therein without departing from the spirit and scope of the invention.

The embodiments of the invention in which an exclusive property or privilege is claimed are defined as follows:

1. A method for the removal of a film adhered to skin, comprising:
rubbing the film with a microfiber article wetted with a removal formulation.

2. The method of claim 1, further comprising soaking the film for period of time with the wetted microfiber article before rubbing.

3. The method of claim 2, wherein soaking comprises about 30 seconds or less.

4. The method of claim 1, wherein the microfiber article is a mitten.

5. The method of claim 1, wherein the removal formulation is an oil-based formulation

6. The method of claim 1, wherein the removal formulation is a water-based formulation.

7. The method of claim 1, wherein the removal formulation is a biphasic formulation comprising an oil and water phase.

8. The method of claim 1, wherein the film is a skin tightening formulation.

9. The method of claim 1, wherein the film is adhered on a facial feature.

10. The method of claim 9, wherein the facial feature is on or near the eye.

11. The method of claim 1, wherein rubbing comprises rubbing for under 2 minutes to remove the film.

12. A combination of an article and formulation, comprising:
an article having at least one exterior surface section made of microfibers; and
a removal formulation configured for removing a film adhered on skin.

13. The combination of claim 12, wherein the article is a mitten.

14. The combination of claim 12, wherein the exterior of the article comprises a fabric made from microfibers of less than 1 denier.

15. The combination of claim 12, wherein the interior of the article is lined with an impermeable layer.

16. The combination of claim 12, wherein the exterior of the article comprises a terry cloth fabric made from microfibers of less than 1 denier.

17. The combination of claim 12, wherein the exterior of the article is made from microfibers that have a cross sectional shape having corners.

18. The combination of claim 12, wherein the exterior of the article is made from polyester or polyamide or both polyester and polyamide microfibers.

19. The combination of claim 12, wherein the removal formulation is an oil-based formulation.

20. The combination of claim 12, wherein the removal formulation is a water-based formulation.

21. The combination of claim 12, wherein the removal formulation is a biphasic formulation having an oil and water phase.

22. The combination of claim 12, further comprising a package containing the microfiber article and the removal formulation.
